(19) United States
(12) Patent Application Publication
(10) Pub. No.: US 2005/0208479 A1
(43) Pub. Date: Sep. 22, 2005

(54) Human antibodies for use as a therapeutic agent against vaccinia or small pox

(76) Inventors: Katherine S. Bowdish, Del Mar, CA (US); Martha A. Wild, San Diego, CA (US)

Correspondence Address: CDFS - 1087, 445 BROAD HOLLOW RD, SUITE 225, MELVILLE, NY, 11747, US

(21) Appl. No.: 10/504,386

(22) PCT Filed: Feb. 10, 2003
(86) PCT No.: PCT/US03/03880

§ 371 Date: Apr. 22, 2005

Related U.S. Application Data
(60) Provisional application No. 60/356,087, filed on Feb. 11, 2002.

(51) Int. Cl. C12Q 1/70

(52) U.S. Cl. 435/5; 530/388.3

(57) ABSTRACT

Fully human antibodies or antibody fragments have a binding affinity to one or more vaccinia or variola antigens and the ability to neutralize the virus.

### BACKGROUND

1. Technical Field

The present disclosure relates generally to human antibodies useful against the effects of vaccinia or variola virus (small pox) and, more particularly, to the identification of human antibodies which bind to or sterically hinder the virus to prevent cellular infection.

2. Background of Related Art

For centuries, vaccinia virus has been used to protect man against small pox, and is still the best preventive treatment available. Use of the vaccine in the general public has been discontinued, however, due to the small but real risk of adverse reactions, including death. In addition, the AIDS epidemic has increased the difficulty of reintroducing smallpox vaccination, since immune-compromised patients can be seriously affected by exposure to vaccinia. Vaccinia strains that have been further attenuated (such as, for example, the NYVAC strain designed by Virogenics), have been developed over the years in the course of designing recombinant vaccines to other illnesses. See Virology, vol. 188, pages 217-232 (1992). These strains might be of use if a new vaccine campaign was undertaken.

An excellent review of small molecule inhibitors of vaccinia virus can be found in Clin. Microbiol. Rev., April 2001, pages 382-397. A number of agents are presented therein which have been shown to have some efficacy against vaccinia in vitro or in vivo in animal models. Unfortunately, many of these compounds are only useful for prophylaxis if given before or immediately after exposure to vaccinia. Only cidofovir has actually seen limited use in humans, where it has been tried in AIDS patients with other pox virus diseases, and where it was efficacious. In several animal models, cidofovir required only one dose, and could protect even when given several days after infection, during illness. At present this would be the only therapeutic available for small pox infection in the event of an outbreak.

Neutralization of the related vaccinia virus in vitro and in vivo with polyclonal antibodies has been shown to occur. See Virology, vol. 254, pages 71-80 (1999); Virology, vol. 280, pages 132-142 (2001). Human Fab libraries have been generated from immunized donors against vaccinia virus and a number of Fabs which could neutralize vaccinia virus in vitro were identified, which cross-reacted with monkey-pox virus in ELISA. See Virology, vol. 258, pages 189-200 (1999). Unfortunately, those antibodies were not designed to be specific to any particular vaccinia antigen, but rather were panned against a lysate containing a wide variety of molecules.

Though extracellular enveloped viruses (EEV) make up a small portion of virus during the infectious cycle, they are apparently responsible for the widespread dissemination of the virus in vivo, and protection is associated with immune responses to the EEV proteins. A number of different EEV proteins, in particular the A33R, B5R and L1R gene products, have been suggested as being targets for inhibition of infection based on animal models. See, U.S. Published Application 20020009447A1, the disclosure of which is incorporated herein by reference. A33R, though it appears to give the best protection as a protein product, apparently accomplishes this through a non-neutralizing mechanism, as protection does not correlate with antibody titers. The L1R gene product, a myristylated protein, is located in intracellular mature virus (IMV). Use of L1R alone or in combination with A33R as a vaccine can produce partial protection in mice. The primary focus of neutralization appears to be the B5R gene product (gp42, complement activation regulator superfamily). This gene product is found only on the extracellular envelope of the vaccinia virus as opposed to the IMV. However, it is not known whether a neutralizing antibody to B5R alone would protect against smallpox infection.

In the absence of vaccination, our ability to treat smallpox infections has been limited, and it would be desirable to provide some form of therapeutic against vaccinia and/or variola virus.

### SUMMARY

Fully human antibodies against natural or recombinant vaccinia or Variola antigens (such as, for example, B5R, A33R, variola B7R, a chimeric B5R/B7R gene product or any mutant isoforms) are described. The human antibodies are selected from an antibody library. The library is preferably generated from an immunized human source. In particularly useful embodiments, the human antibodies have an affinity of at least 1×10⁻⁸ M for a vaccinia or variola EEV protein and neutralize the virus.

### DETAILED DESCRIPTION OF PREFERRED EMBODIMENTS

The human antibodies in accordance with this disclosure can be whole antibodies or antibody fragments. The antibodies can be heterodimeric or single chain antibodies. The term “heterodimeric” means that the light and heavy chains of the antibody or antibody fragment are bound to each other via disulfide bonds as in naturally occurring antibodies. Single chain antibodies have the light and heavy chain variable regions of the antibody connected through a linker sequence.

The present human antibodies are identified by screening an antibody library. Techniques for producing and screening an antibody library are within the purview of one skilled in the art. See, Rader and Barbas, Phage Display, A Laboratory Manual, Cold Spring Harbor Laboratory Press, Cold Spring Harbor, N.Y. (2000), U.S. Pat. No. 6,291,161 to Lerner et al. and copending U.S. Provisional Application Nos. 60/323,455 and 60/323,400, the disclosures of which are incorporated herein in its entirety by this reference.

Generally, the first step in producing an antibody library in accordance with this disclosure involves collecting cells from an individual that is producing antibodies against one or more vaccinia or variola antigens, such as, for example, viral EEV proteins. Typically, such an individual will have been exposed to a virus. Cells from tissue that produce or contain antibodies are collected from the individual about 7 days after infection or immunization. Suitable tissues include blood and bone marrow.

Once the cells are collected, RNA is isolated therefrom using techniques known to those skilled in the art and a combinatorial antibody library is prepared. In general, techniques for preparing a combinatorial antibody library involve amplifying target sequences encoding antibodies or portions thereof, such as, for example the light and/or heavy chains using the isolated. RNA of an antibody. Thus, for example, starting with a sample of antibody mRNA that is naturally diverse, first strand cDNA can be produced to provide a template. Conventional PCR or other amplification techniques can then be employed to generate the library.

Screening of the antibody library can be achieved using any known technique such as, for example, by panning against a desired viral antigen. In this manner, antibodies that bind to B5R, B7R, A33R or a B5R/B7R chimera can be identified. See Rader and Barbas, Phage Display, A Laboratory Manual, Cold Spring Harbor Laboratory Press, Cold Spring Harbor, N.Y. (2000). Certain vaccinia antigens have been cloned and can be produced recombinantly for use as immunogens. Both vaccinia (several strains) and variola virus have been sequenced. Thus, the expression of recombinant EEV proteins can be readily achieved. For example, the B5R gene from vaccinia has been cloned and expressed in a baculovirus system minus its C-terminal membrane domain. B7R is the variola ortholog of the vaccinia B5R, and shares 92.7% identity with it. The cloned B5R gene can be easily modified in the critical epitope regions so that it more closely resembles B7R. In addition to B5R and 67R, a chimeric B5R/B7R protein can be readily prepared. Those antibodies which have a binding affinity of at least 1×10⁻⁸ M are isolated and tested for neutralizing ability. Neutralizing ability can be assessed in cellular assays that determine the ability of the antibody to block the binding of the virus with cellular receptors. For example, neutralizing assays using 143B tk-cells or inhibition of comet formation can be used to assess viral inhibition as described in Virology, vol. 254, pages 71-80 (1999). Once antibodies having a binding affinity greater than 1×10 M and in vitro neutralizing ability are identified, they can be tested in vivo in animal models, such as, for example the lethal challenges described in Virology, vol. 254, pages 71-80 (1999).

Antibodies identified in this manner advantageously provide an effective treatment for vaccinia or variola infection. Because the present antibodies are fully human antibodies, they are safe and easily tolerated. In addition, multiple doses can be given without rapidly raising an anti-idiotype response. Where full length antibodies are used, the higher affinity and larger size (compared to single chain antibodies) may be preferred because they provide greater residence time within the patient's system.

The route of antibody administration is in accord with known methods, e.g., injection or infusion by intravenous, intraperitoneal, intracerebral, intramuscular, subcutaneous, intraocular, intraarterial, intrathecal, inhalation or intralesional routes, or by sustained release systems. The antibody is preferably administered continuously by infusion or by bolus injection. One may administer the antibodies in a local or systemic manner.

The present antibodies may be prepared in a mixture with a pharmaceutically acceptable carrier. Techniques for formulation and administration of the compounds of the instant application may be found in “Remington's Pharmaceutical Sciences,” Mack Publishing Co., Easton, Pa., latest edition. This therapeutic composition can be administered intravenously or through the nose or lung, preferably as a liquid or powder aerosol (lyophilized). The composition may also be administered parenterally or subcutaneously as desired. When administered systematically, the therapeutic composition should be sterile, pyrogen-free and in a parenterally acceptable solution having due regard for pH, isotonicity, and stability. These conditions are known to those skilled in the art.

Pharmaceutical compositions suitable for use include compositions wherein one or more of th present antibodies are contained in an amount effective to achieve their intended purpose. More specifically, a therapeutically effective amount means an amount of antibody effective to prevent, alleviate or ameliorate symptoms of disease or prolong the survival of the subject being treated. Determination of a therapeutically effective amount is well within the capability of those skilled in the art, especially in light of the detailed disclosure provided herein. Therapeutically effective dosages may be determined by using in vitro and in vivo methods.

While the above description contains many specific details of methods in accordance with this disclosure, these specific details should not be construed as limitations on the scope of the invention, but merely as exemplifications of preferred embodiments thereof. Those skilled in the art will envision many other possible variations that all within the scope and spirit of the invention as defined by the claims appended hereto. Thus, the foregoing description should be viewed as illustrative, not limiting.

1. A fully human antibody or antibody fragment having a binding affinity of at least 1×10⁻⁸ M to one or more antigens selected from the group consisting of vaccinia extracellular enveloped virus proteins, variola extracellular enveloped virus proteins and a B5R/B7R chimeric protein and the ability to neutralize vaccinia virus.

2. A fully human antibody or antibody fragment as in claim 1 which binds to an antigen selected from the group consisting of B5R, A33R and variola B7R.

3. A fully human antibody or antibody fragment as in claim 1 which is a single chain antibody.

4. A fully human antibody or antibody fragment as in claim 1 which is a heterodimeric antibody.

5. A fully human antibody or antibody fragment as in claim 1 which is an antibody fragment.

6. A method for identifying an antibody comprising:
preparing a combinatorial library using RNA isolated from cells obtained from a human subject producing antibodies against to one or more antigens selected from the group consisting of vaccinia extracellular enveloped virus proteins, variola extracellular enveloped virus proteins and a B5R/B7R chimeric protein;
screening the combinatorial library for an antibody having a binding affinity of at least 1×10⁻⁸ M to one or more antigens selected from the group consisting of vaccinia extracellular enveloped virus proteins, variola extracellular enveloped virus proteins and a B5R/B7R chimeric protein and the ability to neutralize vaccinia virus.

7. A method as in claim 6 wherein the step of screening the combinatorial library for an antibody identifies an antibody which binds to an antigen selected from the group consisting of B5R, A33R and variola B7R.

8. A method for preparing a combinatorial library comprising:
obtaining cells from a human subject producing antibodies against to one or more antigens selected from the group consisting of vaccinia extracellular enveloped virus proteins;
isolating RNA from said cells; and
amplifying sequences of said RNA encoding at least a portion of an antibody against to one or more antigens selected from the group consisting of vaccinia extracellular enveloped virus proteins.

9. A pharmaceutical composition comprising:
a fully human antibody or antibody fragment having a binding affinity of at least 1×10⁻⁸ M to one or more antigens selected from the group consisting of vaccinia extracellular enveloped virus proteins, variola extracellular enveloped virus proteins and a B5R/B7R chimeric protein and the ability to neutralize vaccinia virus; and
a pharmaceutically acceptable vehicle.

10. A pharmaceutical composition as in claim 9 wherein the fully human antibody or antibody fragment binds to an antigen selected from the group consisting of B5R, A33R and variola B7R.

11. A pharmaceutical composition as in claim 9 wherein the fully human antibody or antibody fragment is a single chain antibody.

12. A pharmaceutical composition as in claim 9 wherein the fully human antibody or antibody fragment is a heterodimeric antibody.

13. A pharmaceutical composition as in claim 9 wherein the fully human antibody or antibody fragment is an antibody fragment.
